(19) United States
(12) Patent Application Publication
(10) Pub. No.: US 2023/0404972 A1
(43) Pub. Date: Dec. 21, 2023

(54) INHIBITION OF CASPASE PATHWAY AS A TREATMENT FOR LYSOSOMAL STORAGE DISORDERS

(71) Applicant: Lysosomal and Rare Disorders Research and Treatment Center, Inc., Fairfax, VA (US)

(72) Inventors: Renuka Pudi LIMGALA, Fairfax, VA (US); Ozlem GOKER-ALPAN, Fairfax, VA (US)

(73) Assignee: Lysosomal and Rare Disorders Research and Treatment Center, Inc., Fairfax, VA (US)

(21) Appl. No.: 18/254,329

(22) PCT Filed: Nov. 24, 2021
(86) PCT No.: PCT/US2021/060834

§ 371 Date: May 24, 2023

Related U.S. Application Data
(60) Provisional application No. 63/231,418, filed on Aug. 10, 2021.
(60) Provisional application No. 63/231,421, filed on Aug. 10, 2021.
(60) Provisional application No. 63/118,201, filed on Nov. 25, 2020.

(51) Int. Cl. A61K 31/4025; A61K 31/551; A61K 38/07; A61K 38/06; C12Q 1/6883; G01N 33/68; G01N 33/573

(52) U.S. Cl. CPC A61K 31/4025; A61K 31/551; A61K 38/07; A61K 38/06; G01N 2333/924; G01N 33/6863; G01N 33/573; G01N 2333/54; C12Q 1/6883

(57) ABSTRACT

Disclosed is a method of preventing, treating or reducing a lysosomal storage disorder (LSD) or a symptom thereof in a subject in need thereof comprising administering a therapeutically effective amount of caspase inhibitor to the subject. The therapeutically effective amount of caspase inhibitor may be a medicament or a composition for preventing, treating or reducing a lysosomal storage disorder (LSD) or a symptom thereof in a subject comprising a caspase inhibitor, and optionally a pharmaceutically acceptable carrier. One preferred caspase inhibitor is the pan caspase inhibitor Emricasan.

This Application claims the benefit of priority to U.S. Provisional application 63/118,201 filed Nov. 25, 2020, U.S. Provisional application 63/231,418 filed Aug. 10, 2021, and U.S. Provisional application 63/231,421 filed Aug. 10, 2021. All patents, patent applications and publications mentioned in this disclosure are hereby incorporated by reference in their entirety as if each individual publication was specifically and individually indicated to be incorporated by reference.

### BACKGROUND

Lysosomal storage diseases, also referred to herein as Lysosomal storage disorders or LSDs, are a group of more than 50 rare inherited disorders that result from defects in function of lysosomal protein or other lysosomal functions. LSDs are caused by mutations in the genes encoding lysosomal enzymes, enzymatic cofactors, accessory proteins, membrane transporters or trafficking proteins. These genetic mutations lead to a deficiency in the metabolism of lipids, glycoproteins or so-called mucopolysaccharides. Individual LSDs occur with frequencies of about 1:10,000 to 1:250,000. As a group, the incidence of all types of LSD is about 1:5,000. Most of these disorders are autosomal recessively inherited; however, a few are X-linked inherited, such as Fabry disease and Hunter syndrome (MPS II). Although each disorder results from different gene mutations that translate into a deficiency in enzyme activity, they all share a common biochemical characteristic. Nearly all lysosomal disorders originate from an abnormal accumulation of substances inside the lysosome.

LSDs are classified depending on the substrate involved as lipid storage disorders (sphingolipidoses, gangliosidoses, leukodystrophies), mucopolysaccharidoses, glycoprotein storage disorders, mucolipidoses and cystinosis. Other classifications include underlying disease mechanisms or the type of defective enzyme (Platt, d'Azzo, Davidson, Neufeld, & Tifft, 2018). Detailed classification of various LSDs is shown in Table 1 and the paragraph after Table 1.

TABLE 1
A sample list of lysosomal storage disorders classification according to
the stored substrate or material. Entries are separated by semicolons.

| | |
|---|---|
| Glycogen: | Pompe disease; Danon disease; |
| Lipid: | Niemann-Pick disease type C; Wolman disease; |
| Monosaccharide/amino | Free sialic acid storage disorders (Salla disease) |
| acid monomer: | |
| Mucolipidoses: | Mucolipidosis type II; Mucolipidosis type III; Mucolipidosis type |
| | IV |
| Mucopolysaccharidoses | MPS type I (Hunter, Hurler-Schie, Schie); MPS type II (Hunter); |
| (MPS): | MPS type III (San Filippo type A, B, C & D); MPS type IV |
| | (Morquio); MPS type VI (Maroteaux-Lamy); MPS type VII (Sly); |
| | MPS IX (Natowicz) |
| Multiple enzyme | Multiple sulfatsase deficiency; Galactosialidosis |
| defects: | |
| Neuronal Ceroid | CLN1, CLN2, CLN3, CLN4, CLN5, CLN6, CLN7, CLN8, CLN9, |
| Lipofuscinosis: | CLN10, CLN11, CLN12, CLN 13, CLN14 |
| Oligosaccharidoses | Alpha-mannosidosis; Beta-Mannosidosis, Fucosidosis; Schindler |
| (Glucoproteinosis): | disease; Aspartyglucosaminuria; Sialidosis type I; Sialidosis type II |
| | (Mucolipidosis type I) |
| Peptide: | Pycnodysostosis; Cystinosis |
| Sphingolipidoses: | GMI gangliosidosis; GM2 Gangliosidosis; Gaucher disease; |
| | Niemann-Pick type A & B; Farber disease; Farber |
| | lipogranulomatosis; Krabbe (globoid cell leukodystrophy); |
| | Metachromatic leukodystrophy |
| Multiple sphingolipids: | Prosaposis deficiency |

A listing of LSDs, denoted by (1) at least one of the common name of the LSD; (2) detective protein; (3) accumulated substrate; and (4) mutant gene (separated by semicolons) is as follows. Gangliosidosis generalized GM1 type 1; Beta galactosidase; GM1 gangliosides and non-sialiated derivatives; GLB 1. Krabbe's Disease; Galactoside beta-galactosidase (galactosyl ceramidase); Sphingolipids galactosylceremide and psychosine; Galactocerebrosidase (GALC). Metachromatic leukodystrophy (MLD); Aryl sulfatase A; Sulfatides; ASA. Fabry's disease; Alpha-galactosidase A; Globotriaosylceramide (GL-3 or Gb3); GLA. Gaucher disease; Glucocerebrosidase; Glucocerebroside; GBA, Niemann-Pick disease A; Acid sphingomyelinase (ASM); Sphingomyelin; SMPD1. Niemann-Pick disease B; Acid sphingomyelinase (ASM); Sphingomyelin; SMPD1, Niemann-Pick disease C1; Transmembrane protein; Unesterified cholesterol; NPC1. Niemann-Pick disease C2; Transmembrane protein; Unesterified cholesterol; NPC2. Tay-Sachs Disease; Hexosaminidase A; GM2 ganglioside; HEXA. Sandhoff disease; Hexosaminidase B; GM2 gangliosides; HEXB. GM2-activator deficiency; GM2 activator protein; GM2 gangliosides; GM2A gene. Multiple sulfatase deficiency; FGE (formylglycine-generating enzyme); Mucopolysaccharides and sulfatides; SUMF1. Alpha mannosidosis; Alpha mannosidase; incompletely degraded oligosaccharides; MAN2B1. Schindler's disease; Alpha-N-acetylgalactosaminidase; Glycosphingolipids, Glycoproteins, and Oligosaccharides; NAGA. Aspartylglucosaminuria; Aspartylglycosaminidase; Aspartylglycosamine; AGA. Fucosidosis; Alpha-L-Fucosidase; Fucose linked to the N-acetylglucosamine residue attached to asparagine; FUCA1. Hurler syndrome; Alpha-L iduronidase(IDUA); Heparan and Dermatan sulphates; IDUA. Scheie syndrome; Alpha-L iduronidase(DUA); Heparan and Dermatan sulphates; IDUA. Hurler-Scheie syndrome; Alpha-L iduronidase(IDUA); lieparan and Dermatan sulphates; IDUA, Hunter syndrome; Iduronate 2-sulfatase (IDS); Heparan and Dermatan sulphates; IDS. SanFilippo syndrome A; Heparan N-sulfatase; Heparan sulphate; SGSH. SanFilippo syndrome B; Alpha-Nacetylglucosaminidase; Heparan sulphate; NAGLU. SanFilippo syndrome C; Acetyl CoA:alpha glucosaminide acetyltransferase; Heparan sulphate; HGSNA T. SanFilippo syndrome D; N-acetylglucosamine 6-sulatase; Heparan sulphate; GNS. Morquio syndrome A; N-acetylglucosamine 6-sulatase; Keratan sulphate Chondroitin-6-sulphate; GALNS. Morquio syndrome B; Beta-galactosidase; Keratan sulphate; GLB1. Maroteaux-Lamy syndrome; N-acetylgalactosamine-4-sulfatase; Dermatan sulphate Chondroitin-4-sulphate; Aryl sulfatase B. Sly syndrome; Beta glucuronidase; Dermatan sulphate Heparan sulphate Chondroitin 4,6 sulphate; GUSB. Neuronal Ceroid lipofuscinosis NCL 1 through 14; Catabolic enzymes, other proteins; Heterogenous autofluorescent material; CLN1 through CLN14, Galactosialidosis; Protective proteinicathepsin A (PPCA); Sialyloligosacchardes; CTSA. Infantile sialic acid storage disease; Sialin; Free sialic acid; SLC17A5. Salla disease; Sialin; Free sialic acid; SLC17A5. Sialuria; Uridinediphosphate-N-acetylglucosamine 2-epimerase; Sialic acid; GNE. Sialidosis I and II (Mucolipidosis I); Neuraminidase 1 (NEU1); Salic acid-containing compounds; NEU1. I-cell disease (Mucolipidosis II); GlcNAc-1-phosphotransferase (production reduced); Many large molecules; GNPTAB, Pseudo-Hurler-Polydytrophy (Mucolipidosis III); GlcNAc-1-phosphotransferase (activity reduced); Many large molecules; GNPTAB. Mucolipidosis IV; Lucolipin-1; Lipids and proteins; MCOLN1. Lysosomal acid lipase deficiency infantile and childhood/adult types; Lysosomal Acid lipase; Cholesteryl esters, triglycerides, and other lipids; LIPA. Pompe's disease (Glycogen storage disease type II); Acid alpha-glucosidase (GAA); Glycogen; GAA, Danon disease (glycogen); LAMP2 protein; Glycogen; LAMP2. Cystinosis(cystine); Cystinosin; Cystine; CTNS.

While the symptoms of lysosomal storage disease vary, depending on the particular disorder and other variables like the age of onset, and can be mild to severe, in most the nervous system is involved. Some neurologic presentations include developmental delay, movement disorders, seizures, dementia, deafness and/or blindness. The systemic presentations of Lysosomal storage disease include an enlarged liver (hepatomegaly) and an enlarged spleen (splenomegaly), kidney, pulmonary, eye and cardiac problems, and abnormal development of bones and cartilage. Lysosomal storage diseases affect all ages, and the more severe the disease is, the earlier is the presentation. The most severely affected children often die at a young and unpredictable age, many within a few months or years after birth.

The advent of enzyme replacement therapy (ERT) followed by the development of substrate reduction and pharmacological chaperone therapies enabled different treatment alternatives for some of the common LSDs, including Gaucher disease (GD), Pompe disease (PD) and Fabry disease (FD) (Beck, 2018; Marques & Saftig, 2019). While the current therapies, do alleviate various symptoms, there are limitations as the current treatments do not truly cure the patients, but slow the disease progression and stabilize organ dysfunction (Beck, 2018),

### BRIEF DESCRIPTION

Disclosed is a method of preventing, treating or reducing a lysosomal storage disorder (LSD) or a symptom thereof in a subject in need thereof comprising administering a therapeutically effective amount of caspase inhibitor to the subject (also referred to in this disclosure as “patient”). The therapeutically effective amount of caspase inhibitor may be a medicament or a composition for preventing, treating or reducing a lysosomal storage disorder (LSD) or a symptom thereof in a subject comprising a caspase inhibitor, and optionally a pharmaceutically acceptable carrier.

One embodiment is directed to a method of preventing, treating, or reducing a lysosomal storage disorder (LSD) or a symptom thereof in a subject in need thereof comprising administering a therapeutically effective amount of one or more caspase inhibitors to the subject. The caspase inhibitor may be a pan caspase inhibitor.

The method may further comprise a step of determining that the subject has a lysosomal storage disorder or is at risk for developing a lysosomal storage disorder before the administering step.

The determining step, in any embodiment, may be by determining a mutation in at least one gene that causes or is associated with LSD in the subject.

The gene may be at least one gene selected from the group consisting of: GBA; GLA; GAA; GALC; ASA; SMPD1; NPC1.; NPC2; HEXA; HEXB; GM2A; SUMF1; MAN2B1: NAGA; AGA; FUCA1; IDUA; IDS; SGSH; NAGLU; HGSNA T; GNS; GALNS; GLB1; Aryl sulfatase B; GUSB; CLN1; CLN2; CLN3; CLN4; CLN5; CLN6; CLN7; CLN8; CLN9; CLN10; CLN11; CLN12; CLN13; CLN14; CTSA; SLC17A5; GNE; NEW; GNPTAB; MCOLN1; LIPA; 1LAMP2; and CTNS.

The determining step, in any embodiment, may comprise determining in peripheral blood or in body fluid of the subject a biomarker that is indicative of a lysosomal storage disorder or a risk of lysosomal storage disorder.

The biomarker, in any embodiment, may be selected from the group consisting of: IL-1 beta; IL-18; chitotriosidase; CCL18; ACE; TRAP; and ferritin.

The lysosomal storage disorder, in any embodiment, may be at least one disorder selected from the group consisting of: Pompe disease; Danon disease; Niemann-Pick disease types C; Wolman disease; Free sialic, acid storage disorders (Salla disease); Mucolipidosis type II; Mucolipidosis type III; Mucolipidosis type IV; Mucopolysaccharidoses (MPS) type I (Hunter disease, Hurler-Schie disease, Schie disease); MPS type II (Hunter); MPS type III (San Filippo all types (A, B, C, D or E); MPS type IV (Morquio); MPS type VI (Maroteaux-Lamy); MPS type VII (Sly); MPS IX (Natowicz); Multiple sulfatsase deficiency; Galactosialidosis; Neuronal Ceroid Lipofuscinosis CLN1; Neuronal Ceroid Lipofuscinosis CLN2; Neuronal Ceroid Lipofuscinosis CLN3; Neuronal Ceroid Lipofuscinosis CLN6; Neuronal Ceroid Lipofuscinosis CLN5; Neuronal Ceroid Lipofuscinosis CLN6; Neuronal Ceroid Lipofuscinosis CLN7; Neuronal Ceroid Lipofuscinosis CLN8; Neuronal Ceroid Lipofuscinosis CLN9; Neuronal Ceroid Lipofuscinosis CLN10; Neuronal Ceroid Lipofuscinosis CLN11; Neuronal Ceroid Lipofuscinosis CLN12; Neuronal Ceroid Lipotbscinosis CLN 13; Neuronal Ceroid Lipofuscinosis CLN14; Alpha-mannosidosis; Beta-Mannosidosis; Fucosidosis; Schindler disease; Aspartyglucosaminuria; Sialidosis type I; Sialidosis type II (Mucolipidosis type I); Pycnodysostosis; Cystinosis; GM1 gangliosidosis; GM2 Gangliosidosis; Gaucher disease; Niemann-Pick type A; Niemann-Pick type B; Farber disease; Fabry disease; Farber lipogranulomatosis; Krabbe (globoid cell leukodystrophy); Metachromatic leukodystrophy; Prosaposis deficiency; Tay-Sachs Disease; Sandhoff disease; GM2-activator deficiency; Hurler syndrome; Scheie syndrome; Hurler-Scheie syndrome; Hunter syndrome; SanFilippo syndrome A; SanFilippo syndrome B; SanFilippo syndrome C; SanFilippo syndrome D; SanFilippo syndrome E; Morquio syndrome A; Morquio syndrome B; Maroteaux-Lamy syndrome; Sly syndrome; Infantile sialic acid storage disease; Saila disease; Sialuria; I-cell disease (Mucolipidosis II); Pseudo-Hurler-Polydytrophy (Mucolipidosis III); and Lysosomal acid lipase deficiency.

The caspase inhibitor, in any embodiment, may optionally comprise a pharmaceutically acceptable carrier.

The caspase inhibitor, in any embodiment, may be Emricasan which has a chemical name ((3S)-3-{[(2S)-2-{[2-(2-tert-butylanilino)-2-oxoacetyl]amino}propanoyl]amino}-4-oxo-5-(2,3,5,6-tetrafluorophenoxy)pentanoic acid).

The caspase inhibitor, in any embodiment, may be at least one selected from the group consisting of VX-765 (Belnacasan) N-(4-amino-3-chlorobenzoyl)-3-methyl-L-valyl-N-[(2R,3S)-2-ethoxytetrahydro-5-oxo-3-furanyl]-L-prolinamide; VX-740 (Pralnacasan) (4S,7S)-N-[(2R, 3S)-2-ethoxy-5-oxooxolan-3-yl]-7-(isoquinoline-1-carbonylamino)-6,10-dioxo-2,3,4,7,8,9-hexahydro-1H-pyridazino[1,2-a]diazepine-4-carboxamde; Ac-YVAD-cmk (Acetyl-tyrosine-valine-alanine-aspartate-chloromethyl ketone); and Z-VAD-FMK (Carbobenzoxy-valyl-alanyl-aspartyl-[O-methyl]-fluoromethylketone),

In any embodiment, the caspase inhibitor further comprises a pharmaceutically acceptable carrier.

The method of this disclosure, in any embodiment, may reduce one or more biomarkers in peripheral blood or in a body fluid that is indicative of a LSD or is indicative of a risk of developing LSD.

The one or more biomarker, in any embodiment, may be at least one selected. from the group consisting of: IL-1 beta; IL-18; chitotriosidase; CCL18; ACE; TRAP; and ferritin.

In any embodiment, the administering step may be by oral administration; intravenous administration; systemic administration; topical administration to the skin; topical administration to the mucosal membranes; nasal administration; ocular administration; and inhalation administration.

In any embodiment, the administering step may comprise administering a therapeutically effective amount of caspase inhibitor to a subject wherein the dose is administering 0.1 to 200 mg caspase inhibitor per subject per day; 1 mg to 100 mg caspase inhibitor per subject per day; 3 to 75 mg caspase inhibitor per subject per day; or 5 to 50 mg caspase inhibitor per subject per day.

Another embodiment is directed to a medicament or a composition for preventing, treating or reducing a lysosomal storage disorder (LSD) or a symptom thereof in a subject comprising a caspase inhibitor, and optionally a pharmaceutically acceptable carrier. The lysosomal storage disorder may be at least one selected from any LSD in this disclosure. The LSD may be at least one disorder selected from the group consisting of: Pompe disease; Danon disease; Niemann-Pick disease type C; Wolman disease; :Free sialic acid storage disorders (Salla disease); Mucolipidosis type II; Mucolipidosis type III; Mucolipidosis type IV; Mucopolysaccharidoses (MPS) type I (Hunter disease, Hurler-Schie disease, Schie disease); MPS type II (Hunter); MPS type III (San Filippo type A, B, C or D); MPS type IV (Morquio); MPS type VI (Maroteaux-Lamy); MPS type VII (Sly); MPS IX (Natowicz); Multiple sulfatsase deficiency; Galactosialidosis; Neuronal Ceroid Lipofuscinosis CLN1; Neuronal Ceroid Lipofuscinosis CLN2; Neuronal Ceroid Lipofuscinosis CLN3; Neuronal Ceroid Lipofuscinosis CLN4; Neuronal Ceroid Lipofuscinosis CLN5; Neuronal Ceroid Lipofuscinosis CLN6; Neuronal Ceroid Lipofuscinosis CLN7; Neuronal Ceroid Lipofuscinosis CLN8; Neuronal Ceroid Lipofuscinosis CLN9; Neuronal Ceroid Lipofuscinosis CLN10; Neuronal Ceroid Lipofuscinosis CLN11; Neuronal Ceroid Lipofuscinosis CLN12; Neuronal Ceroid Lipofuscinosis CLN 13; Neuronal Ceroid Lipofuscinosis LN14; Alpha-mannosidosis; Beta-Mannosidosis; Fucosidosis; Schindler disease; Aspartyglucosaminuria; Sialidosis type I; Sialidosis type II (Mucolipidosis type I); Pycnodysostosis; Cystinosis; GM1 gangliosidosis; GM2 Gangliosidosis; Gaucher disease; Niemann-Pick type A; Niemann-Pick type B; Farber disease; Fabry disease; Farber lipogranulomatosis; Krabbe (globoid cell leukodystrophy); Metachromatic leukodystrophy; Prosaposis deficiency; Tay-Sachs Disease; Sandhoff disease; GM2-activator deficiency; Hurler syndrome; Scheie syndrome; Hurler-Scheie syndrome; Hunter syndrome; SanFilippo syndrome A; SanFilippo syndrome B; SanFilippo syndrome C; SanFilippo syndrome D; Morquio syndrome A; Morquio syndrome B; Maroteaux-Lamy syndrome; Sly syndrome; Infantile sialic acid storage disease; Salla disease; Sialuria; I-cell disease (Mucolipidosis H); Pseudo-Hurler-Polydytrophy (Mucolipidosis III); and Lysosomal acid lipase deficiency.

The caspase inhibitor may be at least one selected from the group consisting of Emricasan; VX-765 (Belnacasan) N-(4-arnino-3-chlorobenzoyl)-3-methyl-L-valyl-N-[(2R,3S)-2-ethoxytetrahydro-5-oxo-3-furanyl]-L-prolinamide; VX-740 (Pralnacasan) (4S,7S)-N-[(2R,3S)-2-ethoxy-5-oxooxolan-3-yl]-7-(isoquinoline-1-carbonylamino)-6,10-dioxo-2,3,4,7,8,9-hexahydro-1H-pyridazino[1,2-a]diazepine-4-carboxamide; Ac-YVAD-cmk (Acetyl-tyrosine-valine-alanine-aspartate-chloromethyl ketone); and Z-VAD-FMK (Carbobenzoxy-valyl-alanyl-aspartyl-[O-methyl]-fluoromethylketone).

The medicament or composition, in any embodiment, may reduce one or more biomarker in peripheral blood or in a body fluid that is indicative of a LSD or is indicative of a risk of developing LSD.

The one or more biomarkers, in any embodiment, may be at least one selected from the group consisting of IL-1 beta; IL-18; chitotriosidase; CCL18; ACE; TRAP; and ferritin.

The medicament or composition, in any embodiment, may be administered to a subject at a dosage of 0.1 to 200 mg per day, 1 mg to 100 mg per day, 3 to 75 mg per day, or 5 to 50 mg per day.

In any embodiment, the caspase inhibitor may be a pan caspase inhibitor. A pan caspase inhibitor inhibits on one or more caspases.

In this disclosure, any reference to “in any embodiment” would include, at least, in any of the methods, the medicaments, and/or the compositions disclosed. In the claims, that would mean that each claim may be dependent on any other claim or any of the preceding claims. Any claim or embodiment may be combined with any other claim(s) or embodiment(s) and such a combination is also an embodiment or claim of the disclosure.

### BRIEF DESCRIPTION OF THE FIGURES

FIG. 1 depicts the contribution of inflammasome and caspase activation to chronic inflammation in Lysosomal storage disorders. In FIG. 1, it can be seen that accumulated substrates activate procaspases to cleave Pro-IL-1beta and pro-LI-18 into IL-1beta and IL-18 leading to chronic inflammation.

FIG. 2 panels A, B and C depict inflammasome activity in patients with LSDs undergoing disease-specific treatment using enzyme replacement therapy (ERT). In FIG. 2 panels A, B and C, PBMCs from patients with LSDs (Gaucher disease (GD), Fabry disease (FD), and Pompe disease (PD)) and controls were assayed for Caspase-1 activity. FIG. 2, Panel A depicts Caspase-1 activity within PBMCs as assayed using Caspase Glo-1 inflammasome assay. FIG. 2, Panel B depicts secreted amount of caspase-1 assayed using plasma samples collected from peripheral blood. FIG. 2, Panel C depicts secreted amount of caspase-1 dependent cytokine IL-1 beta assayed using plasma samples collected from peripheral blood.

FIG. 3 panels A, B and C depict secretion of beta and IL-18 after treatment using a pancaspase (Emricasan). FIG. 3 Panel A depicts relative amount of secreted IL-1 beta (ng/ml) in the cell culture supernatant with and without treatment with a pancapase inhibitor (Emricasan) for 6 hr. Assays was performed using ELISA. FIG. 3 Panel B depicts relative amount of secreted IL-18 (ng/ml) in the cell culture supernatant with and without treatment with Emricasan for 6 hr. FIG. 3 Panel C depicts relative activity of Caspase-1 in the PBMCs with and without treatment using a pan caspase inhibitor (Emricasan). All assays were performed with ELISA.

FIG. 4 panels A and B depict CCL18 and chitotriosidase—the biomarkers that monitor macrophage activation in LSDs, improve after treatment with Emricasan. FIG. 4 Panel A depicts relative amount of CCL18 (ng/ml) in the cell culture supernatant with and without treatment with a pan caspase inhibitor (Emricasan) for 6 hr. FIG. 4 Panel B depicts relative activity of secreted Chitotriosidase (nmoles of 4MU released per hr per ml) in the cell culture supernatant with and without treatment with a pan caspase inhibitor (Emricasan) for 6 hr. All assays were performed using ELISA.

FIG. 5 Panels A, B and C depict Inflammasome activity and response to Emricasan in different LSDs. In FIG. 5 panel A, PBMC were derived from GD type 1. In FIG. 5, panel B, PBMC were derived from GD type 3. In FIG. 5, panel C, PBMC were derived from a Niemann-Pick patient. After collection, these freshly isolated PBMC were then treated with increasing concentrations of Emricasan for 6 h and were analyzed using Caspace-Glo-1 inflammasome assay and viability kit V8.

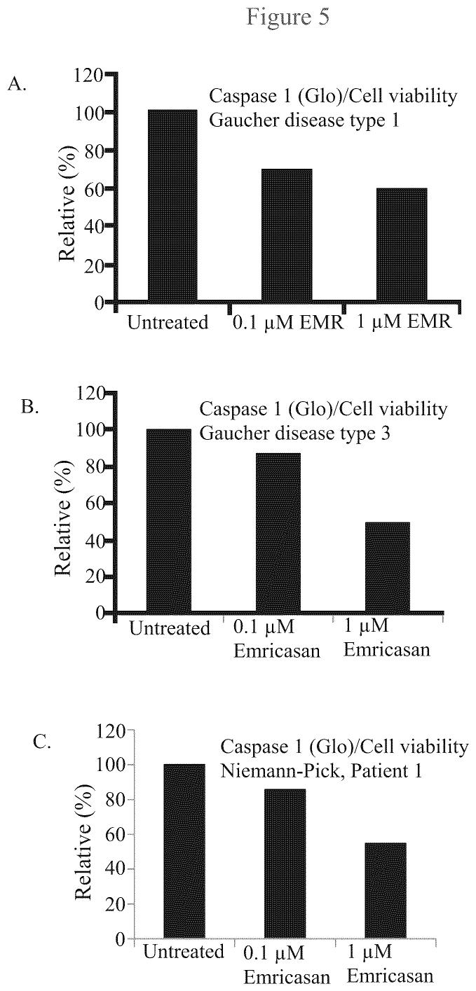

FIG. 6 panels A and B depict secretion of IL-1 beta and IL-18 after treatment with Emricasan in different LSDs. In FIG. 6 Panel A: Relative amount of secreted IL-1 beta in the cell culture supernatant collected after treatment PBMC derived from Gaucher Disease (GD) and Fabry Disease (FD) patients with and without Emricasan for 6 hr was assayed using Il-1 beta ELISA. In FIG. 6 Panel B, relative amount of secreted IL-18 in the cell culture supernatant collected after treatment PBMC derived from GD and ED patients with and without Emricasan for 6 hr was assayed using Il-18 ELISA.

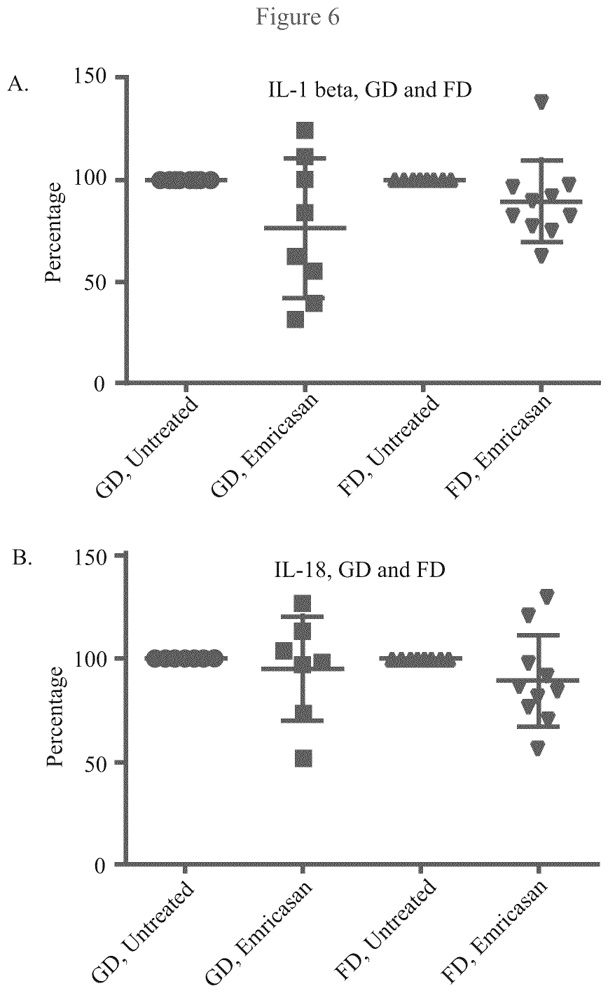

### DETAILED DESCRIPTION

Inflammatory response along with secondary immune activation and dysfunction is the hallmark for all LSDs. Inflammation plays an important role in the pathophysiology of neurodegeneration associated with many neuropathic LSDs, including GM1 gangliosidosis, GM2 gangliosidosis, mucopolysaccharidosis IIIB (Sanfilippo type B), Niemann-Pick type C (NPC), and neuronal ceroid lipofuscinosis (NCL). Alteration of macrophage and microglial cell function impairs the innate immune system, which consequently increases levels of proinflammatory response, including chemokines and cytokines. In addition, dying or damaged cells can activate microglia to initiate an inflammatory response.

### Surrogate Biomarkers to Evaluate and Predict Disease Burden of Iysosomal Storage Disorders

Macrophage activation markers have been used as a biomarker for many LSDs. Macrophages play a critical role in the inflammatory process by producing excessive amounts of proinflammatory cytokines, and drive the tissue damage. Caspase pathway and caspases play an important role in macrophage activation, and in animal models studying sepsis and endotoxic shock, inhibition of caspase pathway has been shown to alleviate the proinflammatory status by promoting Myeloid Derived Stem Cell mediated inhibition of macrophage activation (Li et al., 2019 Front Immunol https://doi.org/10.3389/fimmu2019.01824.

Several molecules released from the activated macrophages have been used as surrogate biomarkers to assess disease activity in LSDs. Chitotriosidase is an enzyme that is secreted from activated in tissue macrophages, and to some extent from the epithelial cells. This property of chitotriosidase makes it a potential biomarker for many disease processes and prognostication. Plasma chitotriosidase level is significantly elevated in Gaucher disease and Niemann-Pick disease type A/B/C and mild to moderately elevated in several other LSDs including GM1 Gangliosidosis, Krabbe, Metachromatic leukodystrophy, Wolmann, Fabry and Morquio diseases. One study looked at plasma chitotriosidase activity in LSDs and concluded that moderately raised activity of chitotriosidase can be utilized as a positive predictive test for certain LSDs (Sheth, 2010). Chronic inflammation and immune activation and dysfunction are observed across multiple LSDs, which persist despite long-term therapy administration (Pandey et al., 2017; Rigante, Cipolla, Basile, Gulli, & Savastano, 2017; Rozenfeld & Fetiozzi, 2017). There are dual actions of inflammatory response, both regulatory and pathogenic. For example, chitriosidase secreted from liver (Kupfer) cells, could induce hepatic fibrosis and cirrhosis (L Malaguarnera et al, Gut 2006). It has been postulated that chronic inflammatory response and proinfl antmatoiy cytokine production due to the stored substrate further drives the substrate production and results in disease progression. The opposite is true, when the inflammation and secondary cytokine production are inhibited pharmacologically in animal models; the mice were protected from the adverse effects of the substrate storage, and survived (Pandey et al., 2017). Hence, a treatment that could effectively target the chronic inflammatory response can be used as a therapeutic modality.

Studying peripheral blood mononuclear cells (PBMCs) has become an important diagnostic tool in lysosomal storage diseases. Studies revealed that subclasses of B and T lymphocytes participate in activation of inflammatory pathways (Limgala et al., 2016; Limgala et al., 2019). When blood samples from patients with different lysosomal storage disorders were studied, disease-specific lysosomal storage material could be found in monocytes suggesting that lysosomal storage in the monocyte-macrophage system might be found in several LSDs (Kieseier, & Goebel, 1997).

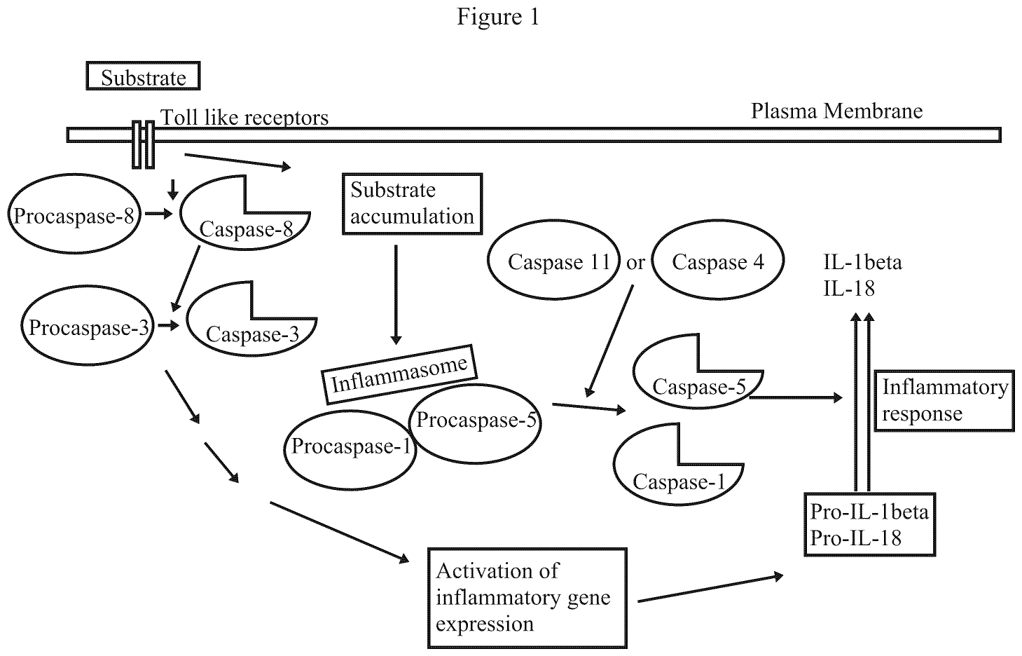

In the case of Gaucher disease, which is the most common LSD, mutations in GBA1 gene result in excessive accumulation of substrate, glucosylceramide in multiple innate and adaptive immune cells in the spleen, liver, lung and bone marrow, often leading to chronic inflammation. Extensive storage of glucosylceramide then induces activation of complement-pathway components that fuels a cycle of cellular substrate accumulation, innate and adaptive immune cell recruitment and activation in Gaucher disease (Pandey et al., 2017). Further, the substrate accumulation leads to impaired lysosomal functions such as autophagy, which is shown to lead to inflammasome activation (Aflaki et al., 2016). In addition to Gaucher disease, heightened caspase-1 mediated inflammasome activity has been seen reflecting clinical significance in several of LSDs including juvenile Batten disease, Cystinosis, GM2 Gangliosidosis, and Niemann-Pick type C disease etc. (Burkovetskaya, Bosch, Karpuk, Fallet, & Kielian, 2019; Li et al., 2005; Matsuoka., Tsuji, Taki, &. Itoh, 2011; Prencipe et al., 2014). FIG. 1 illustrates the current understanding of the mode of action for chronic inflammation in LSDs.

The final common pathway of inflammasome signaling is inflammatory caspase activation. Inflammatory caspases (1, 4, 5, 11) initiate inflammation, that results in inflammatory form of programmed cell death or pyroptosis (Zheng et al., 2020). We have used a pan-caspase inhibitor (Emricasan) in an in vitro cell culture model derived from peripheral blood mononuclear cells from several LSD patients to study the efficacy of such treatment by evaluating caspase specific cytokines (IL-1 beta and IL-18)(Aflaki et al., 2016) as well as immune activation markers- chitotriosidase and CCL18 (Boot et al., 2004; Hollak, van Weely, van Oers, & Aerts, 1994; Raskovalova et al., 2017; Vedder et al., 2006; Veys et al., 2020). Chitotriosidase and other macrophage activation markers including CCL18, ACE, TRAP and Ferritin are routinely used as biomarkers both in clinics and in clinical trials that led to drug development not only as a biomarker of disease activity for Gaucher disease but also as a therapeutic response biomarker. Thus, a decrease in Chitotriosidase with a therapeutic modality is an accepted positive therapeutic response not only by the regulatory agencies (FDA), but also in clinical grounds. While excessive elevation of Chitotriosidase has been shown as a specific biomarker for Gaucher disease, increased Chitotriosidase levels have been shown in other Lysosomal Disorders of varying cellular pathophysiologies such as Fabry disease and Cystinosis, indicating that secondary macrophage activation of varying degrees is universal in LSDs and macrophage activation markers are used as reliable markers of therapeutic response. We demonstrate that addition of a pan-caspase inhibitor significantly reduces the inflammation resulting from inflammasome activation as evidenced by a decrease in IL-1 beta and IL-18. This subsequently leads to a significant decrease in macrophage activation markers, chitotriosidase and CCL18 with an expected favorable therapeutic response.

Current therapies including enzyme replacement therapy and substrate reduction therapy, which rely on reducing the substrate, are still associated with inflammation. Hence, we disclose the use of Caspase inhibitors (including but not limited to Caspase 1 and pan-caspase inhibitors) as a novel therapy in LSDs, We disclose that caspase inhibition will alleviate chronic inflammation and improve disease activity and progression in LSDs listed anywhere in this disclosure including, at least, the LSD shown in TABLE 1.

### Some of the Caspase Inhibitors Including the Generic Nomenclature:

“Emricasan” is a pan-caspase inhibitor and is used as a prototype for our claim. Emricasan refers to a compound of molecular formula: C₂₆H₂₇F₄N₃O₇, (IDN-6556; PF-03491390) with an IUPAC name: of (3S)-3-({N-[{[2-(2-Methyl-2-propanyl) phenyl]amino}(oxo) acetyl]alanyl}amino)-4-oxo-5-(2,3,5,6-tetrafluorophenoxy) pentanoic acid. The structure of Emricasan is as follows:

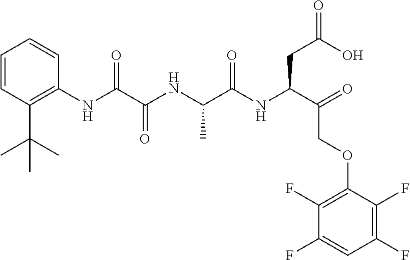 [SMILES: C[C@H](NC(=O)C(=O)Nc1ccccc1C(C)(C)C)C(=O)N[C@@H](CC(=O)O)C(=O)COc1c(F)c(F)cc(F)c1F]

VX-765 (Belnacasan)N-(4-amino-3-chlorobenzoyl)-3-methyl-L-valyl-N-[(2R,3S)-2-ethoxytetrahydro-5-oxo-3-furanyl]L--prolinamide

VX-740 (Pralnacasan) (4S,7S)-N-[(2R,3S)-2-ethoxy-5-oxooxolan-3-yl]-7-(isoquinoline-1-carbonylamino)-6,10-dioxo-2,3,4,7,8,9-hexahydro-1H-pyridazino[1,2-a]diazepine-4-carboxamide

Peptide inhibitors for Caspase 1 include Ac-YVAD-cmk (Acetyl-tyrosine-valine-alanine-aspartate-chloromethyl ketone), Z-VAD-FMK (Carbobenzoxy-valyl-alanyl-aspartyl-[O-methyl]-fluoromethylketone).

In a preferred embodiment, the caspase inhibitor is a pan caspase inhibitor which inhibits one or more caspases.

### METHODS

### Subjects

Subjects with a confirmed diagnosis of one of the LSDs are included in the analysis, Patients (also called subjects in this disclosure) includes at least the following: 1) GD: Patients with Gaucher disease with confirmed disease-causing mutations in GBA gene; 2) FD: Patients with Fabry disease with confirmed disease-causing mutations in GLA gene; 3) PD: Patients with Pompe disease with confirmed disease-causing mutations in GAA gene 4) Patients with Niemann Pick C disease with confirmed disease-causing mutations(s) in NPC 1 or NPC 2 genes and a clinical diagnosis; and 5) Controls: Subjects with no known LSD.

All blood samples are collected after obtaining informed consent according to the internal review board (Western IRB) reviewed protocol (NCT02000310).

### Collection of Plasma

Peripheral blood collected in K2-EDTA tubes was centrifuged at room temperature for 5 minutes at 800 g. Plasma (the upper clear supernatant) was collected into 1.5 ml tubes and frozen at 20° C. till further use. Plasma was used to determine Caspase 1 and cytokine levels as an indication of inherent inflammation in samples.

### Isolation and Culture of Peripheral Blood Mononuclear Cells (PBMCs)

PBMCs are extracted from 3-5 ml peripheral blood using Ficoll-Paque (GE health care). 2-4 ml of whole blood is diluted 1:2 using Phosphate buffered saline (PBS) containing 2% fetal bovine serum (FBS) and overlayed onto Ficoll solution in 15 ml leucosep tube. The tubes are centrifuged at 2000 g for 10 minutes with no brakes. The layer containing PBMCs is transferred into a fresh 15 ml tube and washed with PBS+2% FBS. The cells are then resuspended in RPMI+10% FBS. The cells were then counted using heniocytometer and plated into 96 well plates and set at 37° C. incubator with 5% CO₂.

Treatment of PBMCs with a Pan-Caspase Inhibitor-(3S)-3-({N-[{[2-(2-Methyl-2-propanyl)phenyl]amino}(oxo)acetyl]alanyl}amino)-4-oxo-5-(2,3,5,6-tetrafluorophenoxy) pentanoic acid; Emricasan

The pan-caspase inhibitor, Emricasan (Sigma-Aldrich, Inc., St. Louis, MO, USA), was added to the PBMCs in 96-well plates to a final concentration of 1 μM. After 6 hr of Emricasan treatment, the cells were divided into two groups and used for 1) measurement of Caspase 1 activity within the PBMCs using Caspase-Glo 1 inflammasome assay (Promega, Madison, WI, USA), and 2) the supernatant was collected after centrifuging at 3000 rpm for 5 min to perform ELISA assays for cytokines: IL-1 beta and IL-18 as well as macrophage activation markers: Chitotriosidase and CCL18. The cell culture supernatant was collected into 1.5 ml tubes and stored at 20° C. until further use.

### Assessment of Caspase Inhibition in Relation to Biomarkers Used to Assess and Predict Disease Burden in LSDs

Caspase-1, IL-1 beta and IL-18 were quantified as a measure of capase-1 activity. CCL18 and chitotriosidase were measured as to indicate macrophage activation and reflect clinical outcomes. ELISAs were performed as per manufacturer's protocols to quantify the following cytokines: 1) Caspase 1 (Therrnofisher Scientific, Waltham, MA, USA), 2) IL-1 beta, 3) IL-18 (Abcam, Cambridge, MA, USA), 4) CCL-18 cytokines-1 (Themofisher scientific, Waltham, MA, USA). Chitotriosidase assay was performed using fluorescent substrate 4-Methylumbelliferone chitotrioside (Sigma-Aldrich, Inc., St. Louis, MO, USA).

### Statistical Analysis

All statistical analysis was performed using GraphPad Prism software (GraphPad Software, Inc., La Jolla, CA). Statistical evaluations of the differences were calculated using two-tailed paired t-test, P-values were indicated where found significant, *: P<0.05; **: P<0.01. ***: P<0.001.

### OBSERVATIONS

Elevated Inflammasome Activity in Patients with LSDs

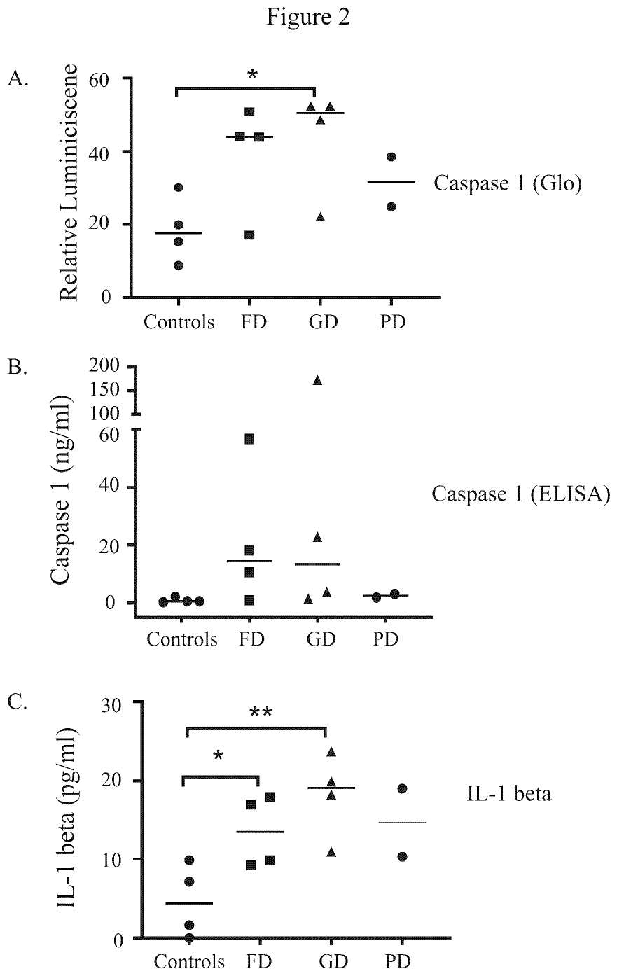

Patients with confirmed diagnosis of any of the LSDs (Gaucher disease (GD), Fabry disease (FD), or Pompe disease (PD)) were assayed for persistent inflammation using inflammasome markers. An increase in Caspase 1 activity within PBMCs was observed in multiple subjects with LSDs compared to non-LSD subjects (FIG. 2, A). Secreted amount of caspase-1 as well as caspase-1 dependent cytokine IL-1 beta were both elevated within the plasma samples of LSD patients and compared to non-LSD subjects (FIG. 2, B & C). The results indicate that patients with LSDs have heightened inflammasome activity despite being under enzyme replacement therapy (ERT) for several years indicating persistent activation of inflammatory pathways.

Treatment with a Caspase Inhibitor Reduces Caspase-1 Mediated Inflammasome Activity

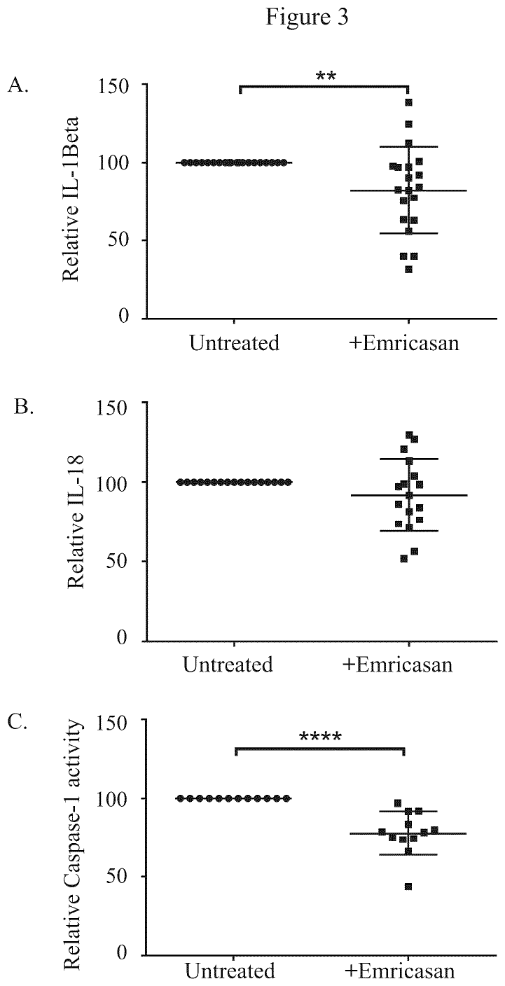

Caspase-1 activity was found to be reduced as a result of treatment of PBMCs with a pan-Caspase inhibitor Emricasan, as seen as a measurement of Caspase 1 activity within the PBMCs using Caspase-Glo 1 inflammasome assay (Promega, Madison, WI, USA) (FIG. 3A). Caspase-1 mediated inflammasome cleaves pro-IL-1 beta and pro-IL-18 into active forms II-1 beta and IL-18 which are secreted from the cells. Hence, we assayed for the amount of IL-1 beta and IL-18 in the cell culture supernatant as an indicator of capase-1 mediated inflammasome activity. PBMCs from LSD patients were isolated and cultured in RPMI+10% FBS. The pancaspase inhibitor, Emricasan was added to a final concentration of 1 uM for 6 hr. The cell culture supernatant was collected from cells without and with the pancaspase inhibitor treatment. A significant decrease in both the interleukins, IL-1 beta and IL-18 was observed after treatment with the pancaspase inhibitor indicating decrease in Caspase-1 activity. Relative decrease for each sample was calculated as relative to untreated PBMCs (FIG. 3A and 3B). Simultaneously, reduction in Caspase-1 activity was assayed as a result to treatment of PBMCs with Pan-Caspase inhibitor as seen. The measurement of Caspase 1 activity within the PBMCs was carried out using Caspase-Glo 1 inflammasome assay (Promega, Madison, WI, USA) (FIG. 3C).

### Pan-Caspase Inhibition Results in Decrease of CCL18 and Chitotriosidase,—the Biomarkers of Macrophage Activation Used for Disease Monitoring and Therapeutic Response in Gaucher Disease and Other LSDs

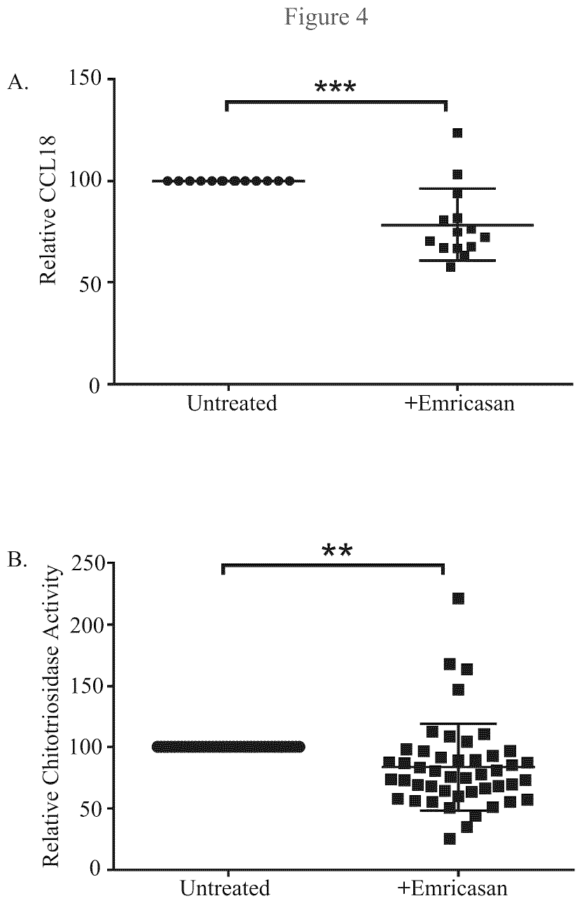

Activated monocytes/macrophages were known to secrete biomarkers, chitotriosidase and CCL18, markers of macrophage activation, which have been used as clinical indicators to study treatment efficacy. Hence, we evaluated the secretion of these biomarkers into cell culture medium in presence of Emticasan. PBMCs from LSD patients when in presence of Emricasan showed a significant decrease in production of both the biomarkers, chitotriosidase and CCL18 (FIG. 4A and B). This indicates that use of Emricasan reduces the macrophage activation with the clinical significance of suggesting that which in turn would control disease activity and progression that occurs secondary to immune activation and dysfunction.

In this specification, stating a numerical range, it should be understood that all values within the range are also described (e.g., one to ten also includes every integer value between one and ten as well as all intermediate ranges such as two to ten, one to five, and three to eight). The term “about” may refer to the statistical uncertainty associated with a measurement or the variability in a numerical quantity that a person skilled in the art would understand does not affect the operation of the invention or its patentability.

All modifications and substitutions that conic within the meaning of the claims and the range of their legal equivalents are to be embraced within their scope. A claim which recites “comprising” allows the inclusion of other elements to be within the scope of the claim; the invention is also described by such claims reciting the transitional phrases “consisting essentially of” (i.e., allowing the inclusion of other elements to be within the scope of the claim if they do not materially affect operation of the invention) or “consisting of” (i.e., allowing only the elements listed in the claim other than impurities or inconsequential activities which are ordinarily associated with the invention) instead of the “comprising” term. Any of these three transitions can be used to claim the invention.

It should be understood that an element described in this specification should not be construed as a limitation of the claimed invention unless it is explicitly recited in the claims. Thus, the granted claims are the basis for determining the scope of legal protection instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the invention. That is, any embodiment or claim may be combined with any other embodiment or claim. Similarly, generalizations of the invention's description are considered to be part of the invention.

From the foregoing, it would be apparent to a person of skill in this art that the invention can be embodied in other specific forms without departing from its spirit or essential characteristics.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

### REFERENCES

Aflaki, E., Moaven, N., Borger, D. K., Lopez, G., Westbroek, W., Chae, J. J., . . . Sidransky, E. (2016). Lysosomal storage and impaired autophagy lead to inflammasome activation in Gaucher macrophages. Aging Cell, 15(1), 77-88. doi:10.1111/ace12409

Beck, M. (2018). Treatment strategies for lysosomal storage disorders. Dev Med Child Neurol, 60(1), 13-18. doi:10.1111/dincri.13600

Boot, R. G., Verhoek, M., de Fost, M., Elollak, C. E., Maas, M., Bleijlevens, B., . . . Aerts, J. M. (2004). Marked elevation of the chemokine CCL18/PARC in Gaucher disease: a novel surrogate marker for assessing therapeutic intervention. Blood, 103(1), 33-39. doi:10.1182/blood-2003-05-1612

Burkovetskaya, M., Bosch, M. E., Karpuk, N., Fallet, R., & Kielian, T. (2019), Caspase 1 activity influences juvenile Batten disease (CLN3) pathogenesis. J Neurochem, 148(5), 652-668. doi:10.1110/jnc.14480

Hollak, C. E., van Weely, S., van Oers, M. H., & Aerts, J. M. (1994). Marked elevation of plasma chitotriosidase activity. A novel hallmark of Gaucher disease. J Clin Invest, 93(3), 1288-1292. doi:10,1172/jci117084

Kieseier, B. C., Wisniewski, K. E., & Goebel, H. H. (1997). The monocyte-macrophage system is affected in lysosomal storage diseases: an immunoelectron microscopic study. Acta Neuropathol, 94(4), 359-362. doi:10.1007/s004010050719

Li, H., Repa, J. J., Valasek, M. A., Beltroy, E. P., Turley, S. D., German, D. C., & Dietschy, J. M. (2005). Molecular, anatomical, and biochemical events associated with neurodegeneration in mice with Niemann-Pick type C disease. J Neuropathol Exp Neurol, 64(4), 323-333. doi 10.1093/jnen/64.4.323

Limgala, R. P., Ioanou, C., Plassmeyer, M., Ryherd, M., Kozhaya, L., Austin, L., . . . Goker-Alpan, O (2016), Time of Initiating Enzyme Replacement Therapy Affects Immune Abnormalities and Disease Severity in Patients with Gaucher Disease. PLoS One, 11 (12), e0168135. doi:10.1371/journal.pone.0168135

Limgala, P., JenneIle, T., Plassmeyer, M., Boutin, M., Lavoie, P., Abaoui, M., . . . Goker-Alpan, O (2019). Altered immune phenotypes in subjects with Fabry disease and responses to switching from agaisidase alfa to agalsidase beta. Am J Transl Res, 11(3), 1683-1696.

Malaguarnera, L. et al., Chitotriosidase gene expression in Kupffer cells from patients with nonalcoholic fatty liver disease. Gut. 2006 Sep; 55(9): 1313-1320.Marques, A. R. A., & Saftig, P. (2019). Lysosomal storage disorders—challenges, concepts and avenues for therapy: beyond rare diseases. J Cell Sci, 132(2). doi:10.1242/jcs.221739

Matsuoka, K., Tsuji, D., Taki, T., & Itoh, K. (2011). Thymic involution and corticosterone level in Sandhoff disease model mice: new aspects the pathogenesis of GM2 gangliosidosis. J Inherit Metab Dis, 34(5), 1061-1068. doi:10.1007/s10545-011-9316-6

Pandey, M. K., Burrow, T. A., Rani, R., Martin, L. J., Witte, D., Setchell, K. D., . . . Grabowski, G. A. (2017). Complement drives glucosylceramide accumulation and tissue inflammation in Gaucher disease. Nature, 543(7643), 108-112. doi:10.11.038/nature21368

Platt, F. M., d'Azzo, A., Davidson, B. L., Neufeld, E. F., & Tifft, C. J. (2018). Lysosomal storage diseases. Nat Rev Dis Primers, 4(1), 27. doi:10,1038/s41572-018-0025-4

Prencipe, G., Caiello, I., Cherqui, S., Whisenant, T., Petrini, S., Emma, F., & De Benedetti, F. (2014). Inflammasome activation by cystine crystals: implications for the pathogenesis of cystinosis. J Am Soc. Nephrol, 25(6), 1163-1169. doi:10.168 1asn.2013060653

Raskovalova, T., Deegan, P. B., Yang, R., Pavlova, E., Stirnemann, J., Labarère, J., . . . Berger, M. (2017). Plasma chitotriosidase activity versus CCL18 level for assessing type I Gaucher disease severity: protocol for a systematic review with meta-analysis of individual participant data. Syst Rev, 6(1), 87. doi:10.1186/s13643-017-0483-x

Rigante, D., Cipolla, C., Basile, U., Gulli, F., & Savastano, M. C. (2017). Overview of immune abnormalities in lysosomal storage disorders. Immunol Lett, 188, 79-85. doi:10.1016/j.imlet.2017.07.004

Rozenfeld, P., & Feriozzi, S. (2017). Contribution of inflammatory pathways to Fabry disease pathogenesis. Mol Genet Metab, 122(3), 19-27. doi:10.1016/j.ymgme.2017.09.004

Vedder, A. C., Cox-Brinkman, J., Hollak, C. E., Linthorst, G. E., Groener, J. E., Helmond, M. T., . . . Aerts, J. M. (2006). Plasma chitotriosidase in male Fabry patients: a marker for monitoring lipid-laden macrophages and their correction by enzyme replacement therapy. Mol Genet Metab, 89(3), 239-244. doi:10.1016/j.ymgme.2006.04.013

Veys, K. R. P., Elmonem, M. A., Van Dyck, M., Janssen, M. C., Cornelissen, E. A. M., Hohenfellner, K., . . . Levtchenko, E. (2020). Chitotriosidase as a Novel Biomarker for Therapeutic Monitoring of Nephropathic Cystinosis. J Am Soc Nephrol, 31(5), 1092-1106, doi:10.1681/asn.2019080774.

### INCORPORATION BY REFERENCE

All publications, patent applications, and patents mentioned herein are hereby incorporated by reference in their entirety as if each individual publication or patent was specifically and individually indicated to be incorporated by reference. In case of conflict, the present application, including any definitions herein, will control.

1. A method of preventing, treating or reducing a lysosomal storage disorder (LSD) or a symptom thereof in a subject in need thereof comprising
administering a therapeutically effective amount of one or more caspase inhibitors to the subject.

2. The method of claim 1, further comprising
determining that the subject has a lysosomal storage disorder or is at risk for developing a lysosomal storage disorder before the administering step.

3. The method of claim 2, wherein the determining step is by determining a mutation in at least one gene that causes or is associated with LSD in the subject.

4. The method of claim 3, wherein the gene is at least one selected from the group consisting of: GBA; GLA; GAA; GALC; ASA; SMPD1; NPC1; NPC2; HEXA; HEXB; GM2A; SUMF1; MAN2B1; NAGA; AGA; FUCA1; IDUA; IDS; SGSH; NAGLU; HGSNA T; GNS; GALNS; GLB1; Aryl sulfatase B; GUSB; CLN1; CLN2; CLN3; CLN4; CLN5; CLN6; CLN7; CLN8; CLN9; CLN10; CLN11; CLN12; CLN13; CLN14; CTSA; SLC17A5; GNE; NEU1; GNPTAB; MCOLN1; LIPA; LAMP2; and CTNS.

5. The method of claim 2, wherein the determining step comprises determining in peripheral blood or in body fluid of the subject a biomarker that is indicative of a lysosomal storage disorder or a risk of lysosomal storage disorder.

6. The method of claim 5, wherein the biomarker is selected from the group consisting of: IL-1 beta; IL-18; chitotriosidase; CCL18; ACE; TRAP; and ferritin.

7. The method of claim 1, wherein the lysosomal storage disorder is at least one disorder selected from the group consisting of: Pompe disease; Danon disease; Niemann-Pick disease types C; Wolman disease; Free sialic acid storage disorders (Salla disease); Mucolipidosis type II; Mucolipidosis type III; Mucolipidosis type IV; Mucopolysaccharidoses (MPS) type I (Hunter disease, Hurler-Schie disease, Schie disease); MPS type II (Hunter); MPS type III (San Filippo all types (A, B, C, D or E); MPS type IV (Morquio); MPS type VI (Maroteaux-Lamy); MPS type VII (Sly); MPS IX (Natowicz); Multiple sulfatsase deficiency; Galactosialidosis; Neuronal Ceroid Lipofuscinosis CLN1; Neuronal Ceroid Lipofuscinosis CLN2; Neuronal Ceroid Lipofuscinosis CLN3; Neuronal Ceroid Lipofuscinosis CLN4; Neuronal Ceroid Lipofuscinosis CLN5; Neuronal Ceroid Lipofuscinosis CLN6; Neuronal Ceroid Lipofuscinosis CLN7; Neuronal Ceroid Lipofuscinosis CLN8; Neuronal Ceroid Lipofuscinosis CLN9; Neuronal Ceroid Lipofuscinosis CLN10; Neuronal Ceroid Lipofuscinosis CLN11; Neuronal Ceroid Lipofuscinosis CLN12; Neuronal Ceroid Lipofuscinosis CLN 13; Neuronal Ceroid Lipofuscinosis CLN14; Alpha-mannosidosis; Beta-Mannosidosis; Fucosidosis; Schindler disease; Aspartyglucosaminuria; Sialidosis type I; Sialidosis type II (Mucolipidosis type I); Pycnodysostosis; Cystinosis; GM1 gangliosidosis; GM2 Gangliosidosis; Gaucher disease; Niemann-Pick type A; Niemann-Pick type B; Farber disease; Fabry disease; Farber lipogranulomatosis; Krabbe (globoid cell leukodystrophy); Metachromatic leukodystrophy; Prosaposis deficiency; Tay-Sachs Disease; Sandhoff disease; GM2-activator deficiency; Hurler syndrome; Scheie syndrome; Hurler-Scheie syndrome; Hunter syndrome; SanFilippo syndrome A; SanFilippo syndrome B; SanFilippo syndrome C; SanFilippo syndrome D; SanFilippo syndrome E; Morquio syndrome A; Morquio syndrome B; Maroteaux-Lamy syndrome; Sly syndrome; Infantile sialic acid storage disease; Salla disease; Sialuria; I-cell disease (Mucolipidosis II); Pseudo-Hurler-Polydytrophy (Mucolipidosis III); and Lysosomal acid lipase deficiency.

8. The method of claim 1, wherein the caspase inhibitor comprises Emricasan ((3S)-3-{[(2S)-2-{[2-(2-tert-butylanilino)-2-oxoacetyl]amino}propanoyl]amino}-4-oxo-5-(2,3,5,6-tetrafluorophenoxy)pentanoic acid).

9. The method of claim 1, wherein the caspase inhibitor comprises at least one selected from the group consisting of VX-765 (Belnacasan) N-(4-amino-3-chlorobenzoyl)-3-methyl-L-valyl-N-[(2R,3S)-2-ethoxytetrahydro-5-oxo-3-furanyl]-L-prolinamide; VX-740 (Pralnacasan) (4S,7S)-N-[(2R,3S)-2-ethoxy-5-oxooxolan-3-yl]-7-(isoquinoline-1-carbonylamino)-6,10-dioxo-2,3,4,7,8,9-hexahydro-1H-pyridazino[1,2-a]diazepine-4-carboxamide; Ac-YVAD-cmk (Acetyl-tyrosine-valine-alanine-aspartate-chloromethyl ketone); and Z-VAD-FMK (Carbobenzoxy-valyl-alanyl-aspartyl-[O-methyl]-fluoromethylketone).

10. The method of claim 1, wherein the caspase inhibitor further comprises a pharmaceutically acceptable carrier.

11. The method of claim 1, wherein the method reduces one or more biomarkers in peripheral blood or in a body fluid that is indicative of a LSD or is indicative of a risk of developing LSD.

12. The method of claim 11, wherein the one or more biomarker is at least one selected from the group consisting of: IL-1 beta; IL-18; chitotriosidase; CCL18; ACE; TRAP; and ferritin.

13. The method of claim 1, wherein administering is by oral administration;
intravenous administration; systemic administration; inhalation administration; topical administration to the skin; topical administration to the mucosal membranes; nasal administration; and ocular administration.

14. The method of claim 1, wherein administering a therapeutically effective amount of caspase inhibitor to a subject is administering 0.1 to 200 mg caspase inhibitor per subject per day; 1 mg to 100 mg caspase inhibitor per subject per day; 3 to 75 mg caspase inhibitor per subject per day; or 5 to 50 mg caspase inhibitor per subject per day.

15. A medicament or a composition for preventing, treating or reducing a lysosomal storage disorder (LSD) or a symptom thereof in a subject comprising a caspase inhibitor, and optionally a pharmaceutically acceptable carrier.

16. The medicament or composition of claim 15, wherein the lysosomal storage disorder is at least one disorder selected from the group consisting of: Pompe disease; Danon disease; Niemann-Pick disease type C; Wolman disease; Free sialic acid storage disorders (Salla disease); Mucolipidosis type II; Mucolipidosis type III; Mucolipidosis type IV; Mucopolysaccharidoses (MPS) type I (Hunter disease, Hurler-Schie disease, Schie disease); MPS type II (Hunter); MPS type III (San Filippo type A, B, C or D); MPS type IV (Morquio); MPS type VI (Maroteaux-Lamy); MPS type VII (Sly); MPS IX (Natowicz); Multiple sulfatsase deficiency; Galactosialidosis; Neuronal Ceroid Lipofuscinosis CLN1; Neuronal Ceroid Lipofuscinosis CLN2; Neuronal Ceroid Lipofuscinosis CLN3; Neuronal Ceroid Lipofuscinosis CLN4; Neuronal Ceroid Lipofuscinosis CLN5; Neuronal Ceroid Lipofuscinosis CLN6; Neuronal Ceroid Lipofuscinosis CLN7; Neuronal Ceroid Lipofuscinosis CLN8; Neuronal Ceroid Lipofuscinosis CLN9; Neuronal Ceroid Lipofuscinosis CLN10; Neuronal Ceroid Lipofuscinosis CLN11; Neuronal Ceroid Lipofuscinosis CLN12; Neuronal Ceroid Lipofuscinosis CLN 13; Neuronal Ceroid Lipofuscinosis CLN14; Alpha-mannosidosis; Beta-Mannosidosis; Fucosidosis; Schindler disease; Aspartyglucosaminuria; Sialidosis type I; Sialidosis type II (Mucolipidosis type I); Pycnodysostosis; Cystinosis; GM1 gangliosidosis; GM2 Gangliosidosis; Gaucher disease; Niemann-Pick type A; Niemann-Pick type B; Farber disease; Fabry disease; Farber lipogranulomatosis; Krabbe (globoid cell leukodystrophy); Metachromatic leukodystrophy; Prosaposis deficiency; Tay-Sachs Disease; Sandhoff disease; GM2-activator deficiency; Hurler syndrome; Scheie syndrome; Hurler-Scheie syndrome; Hunter syndrome; SanFilippo syndrome A; SanFilippo syndrome B; SanFilippo syndrome C; SanFilippo syndrome D; SanFilippo syndrome E; Morquio syndrome A; Morquio syndrome B; Maroteaux-Lamy syndrome; Sly syndrome; Infantile sialic acid storage disease; Salla disease; Sialuria; I-cell disease (Mucolipidosis II); Pseudo-Hurler-Polydytrophy (Mucolipidosis III); and Lysosomal acid lipase deficiency.

17. The medicament or composition of claim 15, wherein the caspase inhibitor is at least one selected from the group consisting of Emricasan; VX-765 (Belnacasan) N-(4-amino-3-chlorobenzoyl)-3-methyl-L-valyl-N-[(2R,3S)-2-ethoxytetrahydro-5-oxo-3-furanyl]-L-prolinamide; VX-740 (Pralnacasan) (4S,7S)-N-[(2R,3S)-2-ethoxy-5-oxooxolan-3-yl]-7-(isoquinoline-1-carbonylamino)-6,10-dioxo-2,3,4,7,8,9-hexahydro-1H-pyridazino[1,2-a]diazepine-4-carboxamide; Ac-YVAD-cmk (Acetyl-tyrosine-valine-alanine-aspartate-chloromethyl ketone); and Z-VAD-FMK (Carbobenzoxy-valyl-alanyl-aspartyl-[O-methyl]-fluoromethylketone).

18. The medicament or composition of claim 15, wherein the medicament or composition reduces one or more biomarker in peripheral blood or in a body fluid that is indicative of a LSD or is indicative of a risk of developing LSD.

19. The medicament or composition of claim 18, wherein the one or more biomarker is at least one selected from the group consisting of IL-1 beta; IL-18; chitotriosidase; CCL18; ACE; TRAP; and ferritin.

20. The medicament or composition of claim 15, wherein the medicament or composition is administered to a subject at a dosage of 0.1 to 200 mg per day, 1 mg to 100 mg per day, 3 to 75 mg per day, or 5 to 50 mg per day.

21. (canceled)
